(19) United States
(12) Patent Application Publication
(10) Pub. No.: US 2009/0281467 A1
(43) Pub. Date: Nov. 12, 2009

(54) Pump Apparatus for Cardiopulmonary Resuscitation Apparatus

(76) Inventor: Allan Hopkins, Mid Glamorgan (GB)

Correspondence Address: Zarian Midgley & Johnson PLLC, University Plaza, 960 Broadway Ave., Suite 250, Boise, ID, 83706, US

(21) Appl. No.: 12/305,598

(22) PCT Filed: Jun. 18, 2007
(86) PCT No.: PCT/GB2007/002260

§ 371 Date: Dec. 18, 2008

(30) Foreign Application Priority Data
Jun. 19, 2006 (GB) 0612145.3

(51) Int. Cl. A61H 31/00; A61M 16/00

(52) U.S. Cl. 601/41; 128/204.18

(57) ABSTRACT

Portable pump apparatus for the provision of pressurised air to a cardiopulmonary resuscitation (CPR) apparatus, the apparatus comprising a housing with an air inlet and an air outlet, and a substantially oil-free pump located within the housing, the substantially oil-free pump having an air inlet in communication with the air inlet of the housing and an air outlet in communication with the air outlet of the housing, such that the pump apparatus is operable to output pressurised air containing less than 1 mg of particulate oil mist per cubic metre of air. Also provided is a cardiopulmonary resuscitation device incorporating such a portable pump apparatus.

### BACKGROUND TO THE INVENTION

The present invention relates to a device for providing pressurised air to a cardiopulmonary resuscitation (CPR) device, and in particular to a chest compression device.

CPR is an emergency first-aid protocol for an unconscious person on whom neither breathing nor pulse can be detected. The medical term for a patient whose heart is stopped is cardiac arrest (also referred to as cardio respiratory arrest), in which case CPR should be used.

CPR involves a combination of mouth-to-mouth rescue breathing and chest compression that keeps oxygenated blood flowing to the brain and other vital organs until a more definitive medical treatment can restore a normal heart rhythm.

Although CPR can be carried out manually, it is an exhausting process for the life saver. In addition, in the situation where the life saver is a paramedic, for example who has arrived on the scene in an ambulance, there may be several other functions to which that paramedic should be attending which would be impossible to carry out whilst also carrying out CPR due to the intensive nature of the life-saving technique.

Therefore, several devices exist in the prior art to allow chest compressions to be carried out automatically. One such device is the LUCAS Chest Compression/Decompression device. This is a gas driven instrument used to deliver manual cardiac compressions to patients who require immediate resuscitation. The device is considered to be more effective than the delivery of cardiac compressions manually by a life saver and is capable of providing 100 chest compressions per minute.

In use, the LUCAS device is strapped around a patient, and located correctly on the chest of a patient before being switched on to begin the automatic compressions. The device is powered by high pressure air or oxygen of medical grade with a supply pressure of 4-6 bar. The device consumes up to 70 litres of air per minute and the supply pressure, including the pressure drop, must not fall below 3.4 bar.

However, these requirements mean that the LUCAS device may only be operated using compressed air or oxygen supplied from tanks, as it has not, to date, been possible to supply such a volume of air per minute, the air being of medical grade quality, using a pump system. Thus, there is a disadvantage that a life-saver must not only carry the LUCAS device to the patient, but must also carry large tanks of compressed air/oxygen with which to operate the device. Furthermore, the tanks of compressed air/oxygen only provide a limited number of minutes of operation of the chest compression device before the air supply will run out.

Therefore, there is a requirement for an alternative source of medical grade air at an appropriate pressure and volume delivery per minute.

### SUMMARY OF THE INVENTION

The present invention seeks to address the problems of the prior art.

Accordingly, a first aspect of the present invention provides a portable pump apparatus for the provision of pressurised air to a cardiopulmonary resuscitation (CPR) apparatus, the apparatus comprising a housing with an air inlet and an air outlet; and a substantially oil-free pump located within the housing, the substantially oil-free pump having an air inlet in communication with the air inlet of the housing and an air outlet in communication with the air outlet of the housing; such that the pump apparatus is operable to output pressurised air containing less than 1 mg of particulate oil mist per cubic metre of air.

The use of the term “substantially oil-free” is intended to include air containing less than 1 mg of particulate oil mist per cubic metre of air, and preferably no more than 0.5 mg of particulate oil mist per cubic metre of air.

Preferably, the pump apparatus is operable to output pressurised air containing no more than 0.5 mg of particulate oil mist per cubic metre of air. In this way, the air being output from the portable pump apparatus will be within the specifications of medical air.

Preferably, the pressurised air output from the portable pump apparatus provides air having less than 10 mg, and more preferably no more than 5.5 mg of carbon monoxide per cubic metre of air, and/or less than 1800 mg, and preferably no more than 900 mg of carbon dioxide per cubic metre and/or very little or no moisture and/or substantially no bacterial contamination. In this way, the quality of the pressurised air provided by the portable pump apparatus may be within the specifications of medical air which are as follows:
less than or equal to 0.5 mg of particulate oil mist per cubic metre of air;
less than or equal to 5.5 mg of carbon monoxide per cubic metre of air;
less than or equal to 900 mg of carbon dioxide per cubic metre of air;
no moisture;
no bacterial contamination.

In addition, medical grade air should be substantially free from toxic products, flammable or toxic vapours, and odours at all points in the delivery system. Although it is not sterile medical grade air that is required from the portable pump apparatus in order to operate a CPR apparatus such as the LUCAS device, the medical grade air must be clean at standard temperature and pressure and should fulfil the requirements set out immediately above.

In one embodiment, the pump is a WOBL pump. This carries the advantage over a piston pump that the pump may be substantially oil-free and therefore capable of producing medical grade air.

In a further embodiment of the present invention, the pump apparatus is operable to deliver pressurised air at a pressure of up to 12 bar. However, typically the pump will be configured to deliver air at 10 bar pressure.

In this way, the portable pump apparatus will be able to deliver air at the required volume of 70 litres per minute.

Preferably, the CPR apparatus to which the portable pump apparatus is delivering pressurised air is a chest compression device, such as a LUCAS device.

It is important that the pump apparatus does not operate at a volume which is considered to be too high, and therefore it is preferred that the apparatus is adapted to deliver pressure at a noise level not exceeding 85 decibels, and preferably not exceeding 75 decibels. This falls within acceptable guidelines for the amount of noise generated by a medical device, in use.

In one embodiment, the apparatus is further provided with a tank located between the air outlet of the pump and the air outlet of the housing. In this way, a volume of air may be retained within the housing such that when the pump is switched on and a LUCAS device is attached to the patient, when the LUCAS device is switched on there is no immediate drop in pressure through the LUCAS device. Instead, the air held within the tank in the interior of the housing of the portable pump apparatus will already be at a suitable pressure such that when the air inlet to the LUCAS device is switched on there is sufficient air volume at sufficient pressure within the housing of the apparatus to allow the LUCAS device to operate within acceptable pressure and time limits.

In one preferred embodiment, the tank has a common air inlet and outlet. In other words, the tank is not provided in-line with respect to the air stream through the portable pump apparatus, but is instead provided as a “branch” from the main air stream through the apparatus.

As an alternative to a tank, any suitable means of storing a volume of air may be used, such as coil piping or the like.

In one embodiment, the apparatus is further provided with an anti-vibration barrier located between the pump and the interior of the housing. Such an anti-vibration barrier may comprise any suitable means to prevent vibration from the pump being transferred to the housing of the portable pump apparatus, such as anti-vibration feet and/or any form of anti-vibration cradle which supports the pump in use, and absorbs vibrations from the pump, thereby preventing the vibrations from being experienced at the surface of the housing of the portable pump apparatus.

In one embodiment, the pump apparatus is further provided with a pressure relief valve located between the pump and the air outlet and operable to release air from the pump apparatus if the pressure exceeds a specified level.

In this way, the pump may be operated to produce pressurised air at a higher pressure than required, excess pressure build-up in the portable pump apparatus being prevented by the opening of the pressure relief valve when the pressure exceeds a specified level. This has the advantage of ensuring that the pressure through the portable pump apparatus and available at the output of the portable pump apparatus never drops below a specified pressure level.

In a further embodiment, the pump apparatus is further provided with a pressure release system which releases pressure from the system when the pump is switched off. For example, the pressure release system may comprise an electrical switch which detects when power to the pump is stopped. This pressure release system will then allow pressure to be released from the portable pump apparatus, thereby preventing dead-heading when the pump is switched back on.

A portable pump apparatus according to the present invention may also be provided with air intake filters and/or air output filters to ensure that the air being provided under pressure by the pump apparatus is of a particular quality, such filters being intended, for example, to remove particulates from the air and/or to remove moisture from the air.

It is important, particularly in the case where pressurised air is being provided to a chest compression device such as the LUCAS device, that the medical grade air contains very little moisture, as the moisture may interfere with the working of the LUCAS device over time. In addition, it is important that the level of particulates in the pressurised air supply to the LUCAS device follows the specification for medical grade air.

The importance of medical grade air for supplying a device such as the LUCAS device is that although the air is not supplied directly to the patient's lungs, the air will be vented to the atmosphere in the vicinity of the patient and thus there will be a concentration of the air output from the LUCAS device potentially in a closed environment, such as the interior of an ambulance, or the like.

The importance in addition that the pump is substantially oil-free and therefore contains a low level of particulate oil mist is that some of the components of the interior of the chest compression device, such as the LUCAS device, are plastic components which may be damaged by oil particulates provided in the pressurised air.

A further aspect of the present invention provides a CPR assembly comprising a portable pump according to a first aspect of the present invention and a CPR device.

Such a CPR device may comprise a chest compression device, such as a LUCAS device, or the like.

### BRIEF DESCRIPTION OF THE DRAWING

An embodiment of the invention will now be described, by way of example only, and with reference to the accompanying drawing, in which:

FIG. 1 is a block diagram showing the components of an embodiment of a portable pump apparatus in accordance with the present invention.

FIG. 1 shows a portable pump apparatus 10 comprising a housing 20, indicated by dashed lines in FIG. 1. Housing 20 is provided with an air inlet 30 and an air outlet 40. A 50.0 micron intake filter 45 is provided at air intake 30 of housing 20, the intake filter 45 being intended to remove microbes of greater than 50.0 microns in dimension. A WOBL pump 50 is provided downstream of intake filter 45. The WOBL pump 50 has been customised in order to maximise its stroke, bore, shaft thickness and the like in order to specifically produce a required amount of air of 70 litres at 4.1 bar pressure, whilst maintaining its status as substantially oil-free and producing medical grade air output suitable for supplying medical grade air to a chest compression device such as a LUCAS device. Pressurised air emerging from WOBL pump 50 proceeds to air outlet 40 in housing 20 where the pressurised air is available for use with a chest compression device, before high pressure air is output from housing 20, it passes through two output filters of 0.5 and 0.2 microns respectively, but in addition also act as moisture traps. In this way, the air is cleaned of particulates of dimensions greater than 0.2 microns, and the moisture is removed from the pressurised air. As mentioned earlier, the presence of moisture in the pressurised air can affect the proper working of the chest compression device. Furthermore, it is essential that the pressurised air is substantially oil-free, such that damage to plastic seals and components within the LUCAS device or other chest compression devices is prevented.

A reservoir 70 is provided between the pump 50 and the two output filters 60, 65. The reservoir has been set at 8 bar (although it will be appreciated that this setting can be altered if desired) to enable significant first surge of air into the LUCAS device in order to prevent pressure dropping on start-up when air is first drawn into the LUCAS device. The whole system is at 8 bar until the LUCAS device is turned on. This additional pressure fives the LUCAS device the pressure surge required at start-up, ensuring that the pressure does not drop below 3.4 bar. The LUCAS device then subsequently runs at its operating pressure of 4.1 bar.

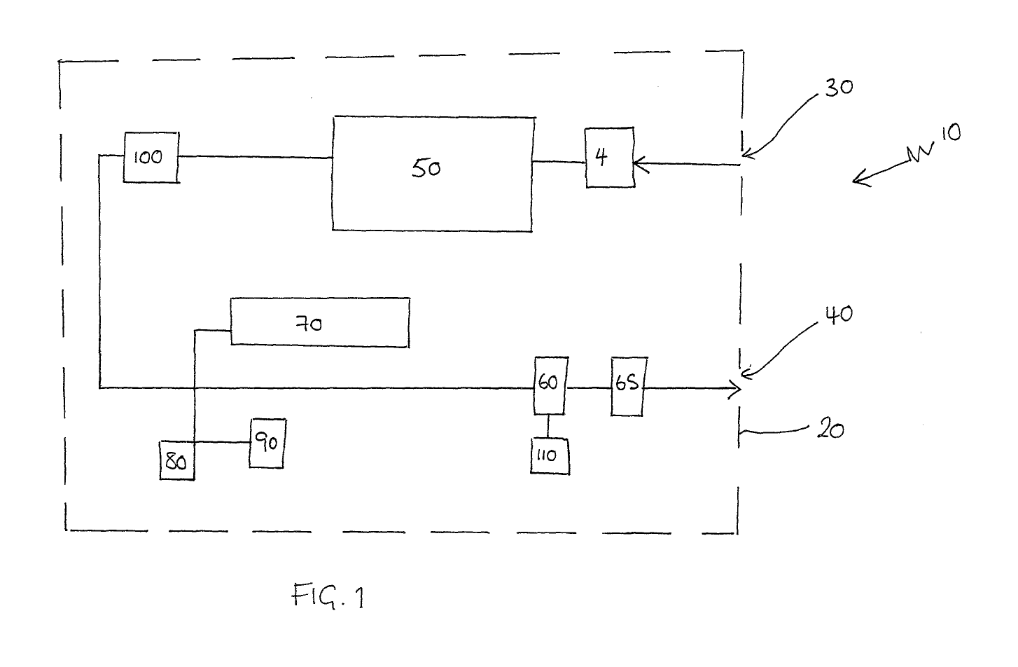

A pressure relief valve 80 is provided between pump 50 and outlet filters 60, 65, the pressure relief valve being set at 8 bar to prevent pump damage when the LUCAS is turned off but the pump remains on. In this way, if pressure builds up within the air system of pump apparatus 10, the pressure will not exceed 8 bar as any rise in pressure above 8 bar will result in the opening of the pressure relief valve 80 in order to vent the pump apparatus system and bring the pressure back to 8 bar. A pressure release system 90 is also provided such that when the pump 50 is turned off, the pump 50 may be immediately turned back on without risk of any “dead-heading” occurring. In the embodiment shown in FIG. 1, the pressure release system 90 is an electrically operated pressure release system which detects when power supply to the pump has been switched off. When the power supply is switched off, the pressure release system is operated to vent the air within the system to the atmosphere. However, it will be appreciated that other forms of pressure release systems could be implemented here in combination with, or as an alternative to, the electrically operated pressure release system of FIG. 1.

Also provided as part of the system of the pump apparatus 10, is a low pressure warning light 100 to draw to the user's attention if the pump apparatus 10 is malfunctioning and not providing the necessary pressure for operating the LUCAS device. In addition, a pressure gauge 110 is provided to allow the actual pressure within the pump apparatus 10 to be monitored throughout operation of the pump apparatus 10.

The overall pump apparatus 10 may weigh less than 15 kilos, and in the particular embodiment discussed with respect to FIG. 1, the total weight is 12.4 kilograms. The pump apparatus 10 is operated under 220/240 volts, 2.7 amps under load, and the housing 20 of pump apparatus 10 has dimensions of approximately 300 mm×300 mm×400 mm. The following information demonstrates the level of performance of the pump apparatus 10 when monitored under testing conditions:
1. The pump apparatus 10 passed emissions testing to EMC Directive 89/336/EEC.
2. The pump apparatus 10 was operated using ambient air and output pressurised air to grade D (breathable air quality), as published by the Compressed Gas Association. The pump apparatus 10 also tested to grade E (as grade D, but with the added species of total hydrocarbons).
3. The following shows the detected exhaust air quality from the pump apparatus 10:

| Analyte | Grade E specification | | Test result | |
|---|---|---|---|---|
| Percentage Oxygen | 20%-22% | | 21.1% | |
| Carbon Monoxide (CO) | <10 | ppm | <1 | ppm |
| Carbon Dioxide (CO₂) | <1000 | ppm | 300 +/− 25 | ppm |
| Oil (condensed droplets) | <5 | mg/m³ | <5 | mg/m³ |
| Odour | No pronounced odour | | No pronounced odour | |
| Total hydrocarbons | <25 | ppm | <0.25 | ppm |
| (as CH₄) | | | | |
| Particles by mass/unit vol | <5 | mg/m³ | <0.1 | mg/m³ |

The testing was carried out by Semtec Laboratories Inc., Phoenix, Ariz. and the quality of the output air from pump apparatus 10 was seen to fall within all required grade E specifications.
4. Vibration and shock testing to 10 g was successful.

Although aspects of the invention have been described with reference to the embodiments shown in the accompanying drawing, it is to be understood that the invention is not limited to the precise embodiments shown and that various changes and modifications may be effected without further inventive skill and effort. It will also be appreciated by the skilled person that although the pump apparatus of the present invention is specifically designed to operate successfully with the LUCAS device and within the specified medical grade air parameters, the pump apparatus of the present invention is also equally applicable to many other CPR devices such as chest compression devices where high pressure air of medical grade is required at high volumes per minute supply.

1. A portable pump apparatus for the provision of pressurised air to a cardiopulmonary resuscitation (CPR) apparatus, the apparatus comprising:
a. a housing with an air inlet and an air outlet;
b. a substantially oil-free pump located within the housing, the substantially oil-free pump having an air inlet in communication with the air inlet of the housing and an air outlet in communication with the air outlet of the housing;
such that the pump apparatus is operable to output pressurised air containing less than 1 mg of particulate oil mist per cubic metre of air.

2. The portable pump apparatus according to claim 1, wherein the pump apparatus is operable to output pressurised air containing no more than 0.5 mg of particulate oil mist per cubic metre of air.

3. (canceled)

4. The portable pump apparatus according to claim 1, wherein the pump apparatus is operable to deliver pressurised air at a pressure of 10 bar.

5. The cardiopulmonary resuscitation assembly according to claim 15, wherein the cardiopulmonary resuscitation device is a chest compression device.

6. (canceled)

7. The portable pump apparatus according to claim 1, wherein the apparatus is adapted to deliver pressurised air at a noise level not exceeding 85 decibels.

8. The portable pump apparatus according to claim 1, wherein the apparatus is further provided with a tank located between the air outlet of the pump and the air outlet of the housing.

9. The portable pump apparatus according to claim 8, wherein the tank has a common air inlet and outlet.

10. The portable pump apparatus according to claim 1, wherein the apparatus is free of electronics.

11. The portable pump apparatus according to claim 1, wherein the apparatus is further provided with an anti-vibration barrier located between the pump and the interior of the housing.

12. The portable pump apparatus according to claim 11, wherein the anti-vibration barrier comprises one or more anti-vibration feet.

13. The portable pump apparatus according to claim 1, wherein the pump apparatus is further provided with a pressure relief valve located between the pump and the air outlet of the housing and operable to release air from the pump apparatus if the pressure exceeds a specified level.

14. The portable pump apparatus according to claim 1, wherein the pump apparatus is further provided with a pressure release system which releases pressure from the apparatus when the pump is switched off.

15. A cardiopulmonary resuscitation assembly comprising a portable pump according to claim 1, and a cardiopulmonary resuscitation device.

16. (canceled)

17. (canceled)
